(19) United States
(12) Patent Application Publication
(10) Pub. No.: US 2008/0140036 A1
(43) Pub. Date: Jun. 12, 2008

(54) Antimicrobial Composition

(76) Inventors: Rachael Buck, Stockport (GB); Michael Edward Donald Crothers, Didsbury (GB); Gordon Nelson, Cheshire (GB)

Correspondence Address: MORRISON & FOERSTER LLP, 12531 HIGH BLUFF DRIVE, SUITE 100, SAN DIEGO, CA, 92130-2040, US

(21) Appl. No.: 11/587,744

(22) PCT Filed: Jan. 18, 2005
(86) PCT No.: PCT/GB05/00128

§ 371 Date: Jan. 25, 2008

(30) Foreign Application Priority Data
Apr. 27, 2004 (GB) 0409373.8

(51) Int. Cl. A61F 13/49; A01N 25/00; A61K 9/50; A61K 31/351; A61K 31/575; A61K 31/7036; A61K 31/4164; A61K 31/155; A61K 31/05; A61K 31/055; A61K 31/505; A61K 31/40; A61K 31/53; A61K 31/428; A01P 3/00; A61P 31/00; A01P 1/00

(52) U.S. Cl. 604/360; 424/404; 514/460; 514/182; 514/40; 514/399; 514/635; 514/729; 514/736; 514/184; 514/424; 514/245; 514/373

(57) ABSTRACT

The present invention relates to a composition for use as an antimicrobial medicament comprising a biocidally active compound. The invention also relates to a medicament comprising at least one biocidally active compound and a fungal cell or fungal cell fragment wherein molecules of the at least one biocidally active compound are encapsulated or partially encapsulated by the fungal cell or fungal cell fragment.

The present invention relates to antimicrobial compositions and methods of using the same. In particular the present invention relates to antimicrobial compositions and methods for preventing and inhibiting microbial growth to control infection, colonization, contamination, biodeterioration and spoilage.

The control of infection, microbial contamination and biodeterioration is mainly achieved by fungicides, bacteriocides (including fungistatic and bacteriostatic agents), anti-parasitic agents and/or antibiotics. However, in high concentrations these synthetic chemicals can be toxic, an irritant or promote allergic responses.

The range of biocides, anti-parasitic agent's, fungicides and bactericides available to the crop protection, industrial and health care sectors is ever dwindling due to ever increasing regulatory pressure.

Furthermore, the development of resistance to these biocides has been observed in many strains of microorganisms. Consequently, many fungicides and bactericides are being phased out by regulatory agencies.

In the healthcare sector, despite major advances preventing and/or treating infection, for example in wound management, infection still remains an important factor in recovery from such afflictions. For instance, in patients with burns, approximately 75% of deaths are due to complications with sepsis from wound infection (1). Among other adverse effects, infection delays healing, contributes to graft failure and can increase the depth of a burn. Approximately 30% of burn wounds become colonized with Staphylococcus aureus (2) and outbreaks of methicillin-resistant S. aureus (MRSA) have created major problems for burn units and intensive care units in terms of cross infection and rehabilitation of the patient due to imposed barrier nursing (3). Some MRSA strains, such as epidemic MRSA (EMRSA) have the ability to spread rapidly among patients and the dominant clonal EMRSA types 15 and 16 are problematic in the UK (4, 5).

Staphylococci are an example of common bacteria that live on the skin and mucous membranes (e.g. in the nose) of humans. About 15-40 percent of healthy humans are carriers of S. aureus, that is, they have the bacteria on their skin without any active infection or disease (colonisation). S. aureus is the most pathogenic species of the Genus as they can cause potentially fatal diseases and currently major concern focuses around their increasing resistance to antibiotics. In the USA and the UK, 90% of S. aureus isolates are resistant to penicillin G and the incidence of methicillin resistance (MRSA) is rising exponentially.

Vancomycin is one of the few effective systemic antibiotics available for treatment, however increased inhibitory concentrations (intermediate resistance) has been reported (Vancomycin intermediate Staphylococcus aureus, VISA) and there is major concern that total antibiotic resistant strains may emerge in the immediate future (6). However, because of the toxicity of vancomycin and the threat of antimicrobial resistance its use is controlled.

To date topical anti-microbial therapy is the single most important component of wound care to prevent infection (7). In hospitalised burns patients, Flamazine™ is by far the most frequently used topical prophylactic agent (8) but this does not always penetrate into the wound (9) and cannot be used to eradicate microbial carriage from the patient or the environment. Thus, a means of preventing infection, reducing microbial colonisation, and reducing the need for administered antibiotics is needed.

Other microbial infections can prove problematic, for example, those caused by Candida albicans. This is a yeast and is normally present on humans as a harmless commensal organism, but can be one of the major fungal pathogens of humans. Infections can be localised, such as vaginal and oral infections, which can cause considerable discomfort. In some patient groups, in particular those who are immunocompromised such as prematurely born infants and leukaemia sufferers, Candida albicans can cause systemic infections that can lead to death. The number of effective treatments is very limited and these treatments can have severe side effects.

Other problems brought about by microbial infection include; topical invasive infections on the skin of an individual, oral infections including dental infections, acne, and foot infections,

Similarly, microbial growth is a major cause of infection and spoilage of many cultivated crops and of plants, causing diseases, for example, moulds, rusts and mildew. Many of these diseases are significant in horticultural systems. The incidence of resistance to many fungicides continues to increase and the level of dosage now required often makes application uneconomic. In many cases the fungicides previously employed have now no significant effect against the target fungus.

For example the most damaging disease in wheat is Septoria tritici and the strobilurin fungicides are failing dramatically due to resistance build-up. Therefore alternative fungicides must be sought or the efficacy of existing one improved.

In the animal healthcare sector, there are many microbial diseases or, conditions which affect livestock of one type or another. Of particular importance are those conditions which can result or contribute to lameness or even death of an animal. Footrot is an infectious disease of livestock including sheep, goats or cattle and it is spread from animal to animal via pasture containing bacteria from the feet of infected animals. Footrot is caused by two different bacteria, Fusobacterium and Bacteroides nodosus of which there are many different strains. Some cause a virulent form of footrot whilst others are less invasive and are termed benign.

Other examples include Digital dermatitis in cattle which is thought to be caused by the bacteria Bacteroides or Treponema but this has not yet been completely established. Conditions of the hoof or foot of the animal are normally treated either by using antibiotics or chemical treatment baths.

Foot or hoof treatments currently being used in foot baths present serious problems of disposal of the chemicals and the pain associated with the treatment. Therefore there exists a need for improved treatments for treating or alleviating many microbial diseases of livestock which are not only less expensive than those currently available but which are also more effective and environmentally benign.

The regulatory process in Europe and the US continues to place restrictions on the use of biocides which come into contact with humans and the environment, in materials, such as film coatings, (in can) paints, plastics, leather, rubber, paper and textiles. The number of molecules available are much reduced

The Biocidal Products Directive (BPD), as implemented in Great Britain under the Biocidal Products Regulations 2001, gives a formal definition of a biocidal product as:
“Active substances and preparations containing one or more active substances, put up in the form in which they are supplied to the user, intended to destroy, deter, render harmless, prevent the action of, or otherwise exert a controlling effect on any harmful organism by chemical or biological means.”

The Directive has a very wide scope, with 23 product types. This covers non-agricultural pesticides currently approved under the Control of Pesticides Regulations 1986 (i.e. wood preservatives, public hygiene insecticides, rodenticides, surface biocides and antifouling paints), as well as a wide range of biocidal products not currently requiring authorisation under other legislation (such as disinfectants, preservatives and a number of other specialist products).

The 23 product types of the Biocidal Products Directive

| | | |
|---|---|---|
| Number | Product Type | Description |

Main Group 1: Disinfectants & General Biocidal Products

| | | |
|---|---|---|
| 1 | Human hygiene | Used for human hygiene purposes. |
| | biological products | |
| 2 | Private and public | Used for the disinfection of air, surfaces, materials, |
| | health area | equipment and furniture which are not used for direct food |
| | disinfectants and other | or feed contact in private, public or industrial areas, |
| | biocidal products | including hospitals, as well as products used as algaecides. |
| | | Usage areas include swimming pools, aquariums, bathing |
| | | and other waters; air-conditioning units; walls and floors in |
| | | health and other institutions; chemical toilets, waste water, |
| | | hospital waste, soil and other substrates (in playgrounds). |
| 3 | Veterinary hygiene | Includes products used in areas in which animals are |
| | biocidal products | housed, kept or transported. |
| 4 | Food and feed area | Used for the disinfection of equipment, containers, |
| | disinfectants | consumption utensils, surfaces or pipework associated with |
| | | the production, transport, storage, or consumption of food, |
| | | feed or drink (including drink water) for humans and |
| | | animals. |
| 5 | Drinking water | For both humans and animals. |
| | disinfectants | |

Main Group 2 Preservatives

| | | |
|---|---|---|
| 6 | In-can preservatives | Used for the preservation of manufactured products, other |
| | | than foodstuffs or feeding stuffs, in containers by the |
| | | control of microbial deterioration to ensure their shelf life. |
| 7 | Film preservatives | Used for the preservation of films or coatings by the control |
| | | of microbial deterioration in order to protect the initial |
| | | properties of the surface of materials or objects such as |
| | | paints, plastics, sealants, wall adhesives, binders, papers, art |
| | | works etc. |
| 8 | Wood preservatives | For wood from and including saw-mill stage, and wood |
| | | products (including preventative and curative products). |
| 9 | Fibre, leather, rubber | Includes the preservation of fibrous materials, such as paper |
| | and polymerised | or textile products. |
| | materials preservatives | |
| 10 | Masonry preservatives | Used for the preservation and remedial treatment of |
| | | masonry or other construction materials other than wood by |
| | | the control of microbiological algal attack. |
| 11 | Preservatives for | Use for the preservation of water and other liquids used in |
| | liquid-cooling and | cooling and processing systems by the control of harmful |
| | processing systems | organisms such as microbes, algae and mussels (not |
| | | drinking water preservation products). |
| 12 | Slimicides | Used for the prevention or control of slime growth on |
| | | materials, equipment and structures, used in industrial |
| | | processes, e.g. on wood and paper pulp, and porous sand |
| | | strata in oil extraction. |
| 13 | Metalworking-fluids | Products used for the preservation of metalworking fluids |
| | preservatives | by the control of microbial deterioration. |

Main Group 3: Pest Control

| | | |
|---|---|---|
| 14 | Rodenticides | Control of mice, rats or other rodents. |
| 15 | Avicides | Control of birds. |
| 16 | Molluscicides | Control of molluscs, e.g. snails that may clog pipes. |
| 17 | Piscicides | Control of fish; excludes products for the treatment of fish |
| | | diseases. |
| 18 | Insecticides, acaricides | e.g. insects arachnids and crustaceans |
| | and to control other | |
| | arthropods | |
| 19 | Repellents or | Used to control, harmful organisms (invertebrates such as |
| | attractants | fleas, vertebrates such as birds), by repelling or attracting, |
| | | including those that are used for human or veterinary |
| | | hygiene either directly or indirectly. |

Main Group 4: Other Biocidal Products

| | | |
|---|---|---|
| 20 | Preservatives for food | Used for the preservation of food or feedstuffs by the |
| | and feedstocks | control of harmful organisms. |
| 21 | Antifouling products | Used to control growth and settlement of fouling organisms |
| | | (microbes and higher forms of plant and animal species) on |
| | | vessels, aquaculture equipment or other structures used in |
| | | water. |
| 22 | Embalming or | Used for the disinfection and preservation of human or |
| | taxidermist fluids | animal corpses, or parts thereof. |
| 23 | Control of vertebrates | i.e. vermin |

The Biocidal Products Directive (BPD) that was implemented in Europe in May, is possibly the most significant piece of legislation to affect the supply and use of biocidal products. The directive will impact all manufacturers, formulators, distributors, importers, and end users of biocidal products. Biocide manufacturers will be required to support their products through a product authorization scheme, which may cost them as much as $5 million for each active product. It is expected that 75 percent of existing biocidally active products will be banned from use in Europe as a result of this new legislation.

The cost of supporting products through the BPD is going to limit the ability of many companies to invest in the research and development of new products.

It is an object of the present invention to alleviate or overcome one or more of the problems associated with the prior art and/or to provide an improved antimicrobial composition. It is a further object of the invention to provide an improved method for inhibiting or preventing microbial development in:

1, wounds or other lesions, on the surface of a substrate and/or a surface of the human or animal body.

### 2. In and on the surface of materials, including paints (in-can and in coatings), plastics, textiles and other biodegradable materials

3. in crops

In accordance with a first aspect of the present invention, there is provided a composition comprising at least one biocidally active compound encapsulated within an adjuvant, wherein the adjuvant comprises a fungal cell or fragment thereof.

The applicants have surprisingly discovered that the biocidally active compound is released from the adjuvant on contact thereof with the microbe. Thus, the present invention provides compositions having improved bioavailability as a result of targeted delivery to the microbes of a microbial infection. Additionally, the applicants have discovered enhanced activity of the encapsulated biocidally active compound when encapsulated.

To enable successful protection from contamination, spoilage and biodeterioration one alternative to introduction of new biocide molecules is to enhance the properties of existing molecules. Many molecules can only be used at low levels and at higher concentrations they are irritant and have to be labelled as such in formulation. Other molecules are sensitive to evaporation, therefore these have to be overloaded to have the desired biocidal effect. Some lipophilic molecules cannot be formulated within aqueous environments due to solubility and bioavailability problems.

The fragment of fungal cell may comprise a fungal cell wall, such as a ghost cell, or a part thereof wherein the part is capable of passively retaining the biocidally active compound.

The term “biocidally active compound” as used herein is meant to include any compound capable of adversely affecting normal functioning of a microbe.

The biocidally active compound may be lipophilic or may comprise a lipophilic moiety. Preferably, the biocidally active compound is lipophilic or substantially lipophilic. The term ‘substantially lipophilic’ as used herein is meant to include those compounds having lipophilic and lipophobic moieties wherein the lipophilic moiety is predominant.

The biocidally active compound may be lipid soluble.

The biocidally active compound may be a fungicide and/or a bactericide, such as, for example antibiotics etc. The biocidally active compound may be selected from phenols and cresols, acids and esters, alkalis, chlorine release agents, iodine compounds, quaternary ammonium compounds, biguanides, diamidines, aldehydes, alcohols, heavy metal derivatives, vapour phase disinfectants, sulphates and nitrites, for example.

The biocidally active compound may comprise one or more essential oils. Essential oils are complex mixtures of odorous, steam volatile or extractable organic compounds, which are synthesised by many types of plant. Essential oils can be found in various parts of a plant, such as the leaves, stem, flowers, cell organelles, fruit, roots, seeds and bark etc. Generally, the principal constituents are aromatic compounds. Each oil may comprise 100-300 compounds

Essential oils most abundant components include one or more Mono-, di- and sesqui-terpenoids (mevalonic acid derived constituents); phenylpropanoids; alkanes (and alkane derivatives, such as alcohols, aldehydes, and carboxylic acids), alkenes, alkynes and derivatives thereof.

Essential oils are typically mixtures of organic aromatic and other compounds that are extractable from plant material by methods such as steam distillation, cold pressing, CO₂ extraction or extraction with organic solvents or any other means known to the person skilled in the art.

Essential oils for use in the present invention include but are not limited to extracts from Bay (Pimenta recemosa); Bergamot (Citrus bergamia); Cardamom (Elettaria cardamom); Cedarwood (Cedrus deodara and Juniperus virginiana); Cinnamon leaf (Cinnamomum zellanicum Ceylon); Clove or clove bud (Eugenia caryophyllata Madagascar extra; Syzygium aromaticum L./Eugenia aromaticum L); Cumin seed (Cuminum cyminum); Eucalyptus (Eucalyptus globulus & radiata); Geranium (Pelargonium graveolens Madagascar bourbon); Grapefruit (Citrus paradisi); Lavender (Lavendula officinalis France); Lemongrass (Cymbopogon citrates); Manuka (Leptospermum scoparium); Marjoram (Origanum majorana); Origanum (Origanum vulgare/Cymbopogon martini); Palmarosa (Origanum heracteoticum); Patchouli (Pogostemon cablin E. India dark); Peppermint (Mentha piperita); Rosemary (Rosmarinus officinalis); Rosewood (Aniba rosaeodora); Sage (Salvia tribola); Sandalwood (Aniba rosaeodora); Savory (Satureia thymbra); Tea Tree (Melaleuca alternifola/Leptospermum petersonii); Thyme (Thymus capitus). Other essential oils useful in the present invention include Sandal oil, KapurTulsi oil, and Ropan oil.

Preferably, compositions according to the present invention comprise one or more essential oils from the group comprising Manuka, Geranium, Lavender, Lemongrass, Tea tree and Rosewood oil. More preferably, the compositions of the present invention comprise two or more essential oils selected from the group comprising Manuka, Geranium, Lavender, Lemongrass, Tea tree and rosewood. More preferably still, the composition of the present invention comprises one or more of the following combinations of essential oils; Rosewood+Manuka, Rosewood+Lemongrass, Rosewood+Geranium, Rosewood+Lavender, Rosewood+Tea tree, Manuka+Lemongrass, Manuka+Geranium, Manuka+Lavender, Manuka+Tea tree, Lemongrass+Tea tree, Lemongrass+Lavender, Lemongrass+Geranium, Geranium+Lavender, Geranium+Tea tree and Lavender and Tea tree.

Other common chemical constituents of essential oils are citral (geranial and neral isomers), limonene, linalyl acetate and estragole (methyl chavicol), mono-, sesqui- and di-terpenoids (mevalonic acid-derived constituents); phenylpropanoids (cinnamic acid-derived compounds) and alkane derivatives (alkanes, alkenes, alkynes, alkanols, alkanals, alkanoic acids: mostly acetogenins).

It is understood that the term “essential oil” as used herein includes the naturally occurring extractable plant oils, mixtures thereof, or one or more of the components found in extractable plant oils, whether naturally or artificially synthesized. The term also includes derivatives and analogues of the components found in extractable plant oils.

The composition preferably contains a biocidally active compound in an amount effective to inhibit the growth of a pathogen on a surface to which the composition is applied. The active ingredient is preferably present in the composition in an amount such that when the composition is applied to a surface, the active ingredient is preferably present in an amount of from about 5 to about 30 μg/cm² on or over said surface.

The biocidally active compound may comprise an essential oil and/or any one or more of the compounds selected from those compounds listed in Table 1 and/or econazole, triclosan, rifampicin and mupirocin.

In one composition, the fungicide is econazole.

In another embodiment, the biocidally active compound may be triclosan (obtainable from Cambiochem California, USA of EMD Biosciences Inc., an affiliate of Merck, Germany

The biocidally active compound may be encapsulated with a carrier. For example, in one embodiment, the biocidally active compound is a crystalline solid soluble in the presence of the carrier. Thus, the carrier may facilitate encapsulation of the biocidally active compound.

The biocidally active compound preferably has a positive partition coefficient (LogP_{o/w}) greater than 0.1, more preferably in the range 0.1-10, even more preferably, 0.5-10, even more preferably still 0.5-7.0, most preferably 2.0-7.0.

The biocidally active compound may have a pH in the range pH1.0-12.0, preferably pH4-9.

Preferably the biocidally active compound is not acidic or basic in nature but if it is acid it should have a pKa between 2.0-7.0, most preferably between 4.0-7.0. If basic it should have a pKa between 7.0-12, most preferably between 7.0-10.0.

Preferably, the biocidally active compound is present in an amount from 1-50 g/100 g of product.

Preferably the biocidally active compound is a liquid at s.t.p (20° C., 1 atm.) or dissolved in an organic solvent. Preferably the biocidally active compound is soluble in the carrier at a level above 10 g/l, preferably above 100 g/l, most preferably above 500 g/l.

The biocidally active compound is preferably in liquid form or solution. This is to facilitate encapsulation within the adjuvant. The biocidally active compound may be liquid in its normal state or it may be a solid, in which case it is preferably dissolved or micro-dispersed in a carrier such as a solvent which is lipid soluble. Suitable carriers include any one or more of the following:
a) primary alcohols within the range C4 to C12, such as nonanol and decanol;
b) secondary and tertiary alcohols;
c) glycols, such as diethylene glycol;
d) esters, particularly esters having straight carbon chains greater than 2 and less than or equal to 12, for example, ethyl butyrate, triacetin;
e) aromatic hydrocarbons such as xylene and acetophenone;
f) any aromatic lipophilic oil with no straight chain branch greater than 12 Carbons; and
g) carboxylic acids between C3 and C12

The carrier is preferably non-miscible with water. Preferably, the carrier is organic and has a molecular weight in the range of 100-700. More preferably, the carrier is not miscible with water.

In one embodiment, the carrier comprises a mixture of 2 or more solvents. Preferably, at least one of the solvents is not miscible with water. More preferably, the mixture of solvents forms a homogeneous liquid mixture.

The carrier may comprise any one or more selected from the following: Alkanes, alkenes, alkynes, aldehydes, ketones, monocyclics, polycyclics, heterocyclics, monoterpenes, furans, pyroles, pyrazines, azoles, carboxylic acids, benzenes, alkyl halides, alcohols, ethers, epoxides, esters, fatty acids, essential oils.

In one embodiment, the carrier may have biocidal activity e.g. benzyl alcohol.

Preferably, the carrier is selected for a particular biocidal compound.

The carrier may comprise any one or more of the following:

TABLE 2
carriers

| Name | logP(o/w) |
|---|---|
| 1-(2-aminophenyl)-1-ethanone | 1.1 |
| Acetophenone (1-phenyl-Ethanone) | 1.7 |
| alpha pinene | 3.9 |
| alpha terpineol | 1.7 |
| Benzene | 2.0 |
| Benzonitrile | 1.5 |
| Benzyl alcohol | 1.1 |
| Bromobenzene | 2.9 |
| 1-butanethiol | 2.1 |
| Butylbenzene | 3.9 |
| caryophyllene | 6.0 |
| Chlorobenzene | 2.6 |
| Cyclohexane | 3.2 |
| Cyclohexanol | 1.6 |
| Decane | 5.3 |
| decanoic acid | 3.5 |
| 5-decanolide | 3.1 |
| Decyl alcohol | 3.8 |
| diallyl disulfide | 3.1 |
| 1,3-Difluorobenzene | 2.4 |
| Dimethyl adipate | 1.4 |
| 3,4-dimethyl phenol | 2.2 |
| 3,7-dimethyl-2,6-octadienal | 1.7 |
| 1,5-dimethyl-1-vinyl-4-hexenyl acetate | 2.7 |
| 1,5-dimethyl-1-vinyl-4-hexenyl hexanoate | 4.5 |
| dipropyl disulfide | 3.7 |
| (+−)-5-dodecanolide | 4.0 |
| dodecanoic acid | 4.4 |
| Epibromohydrin | 2.1 |
| Ethylbenzene | 3.0 |
| ethyl (E)-3-hexenoate | 1.7 |
| 4-ethyl-2-methoxy phenol | 2.4 |
| ethyl 3-methylbutanoate | 1.8 |
| ethyl hexanoate | 2.3 |
| ethyl nonanoate | 3.7 |
| Fluorobenzene | 2.2 |
| Heptane | 3.8 |
| 1-Heptanol | 3.1 |
| heptan-2-one | 1.9 |
| Hexane | 3.3 |
| 1-Hexanol | 2.7 |
| (Z)-3-hexenyl 2-methylbutanoate | 2.8 |
| (Z)-3-hexenyl acetate | 1.5 |
| (Z)-3-hexenyl butanoate | 2.4 |
| 2-hydroxy benzaldehyde | 1.5 |
| indole | 2.3 |
| Iodobenzene | 3.2 |
| 3-Iodotoluene | 3.7 |
| isobutyl phenylacetate | 3.2 |
| 4-isopropyl benzaldehyde | 3.0 |
| 1-isopropyl-4-methylbenzene | 4.0 |
| 5-isopropyl-2-methylphenol | 3.1 |
| 2-isopropyl phenol | 2.7 |
| Limonene (1-methyl-4-(1-methylethenyl)- | 4.8 |
| Cyclohexene | |
| (+)-(S)-1(6),8-P-menthadien-2-one | 1.0 |
| (1R,4R)-8-mercapto-3-P-menthanone | 2.9 |
| Methyl benzoate | 1.8 |
| 3-methyl butylamine | 1.1 |
| 6-methyl quinolene | 2.6 |
| 6-methyl-5-hepten-2-one | 1.0 |
| 6-methyl-5-hepten-2-one | 1.0 |
| 2-methyl hexanoic acid | 2.1 |
| s-methyl 3-methylbutanethioate | 2.1 |
| nonanoic acid | 3.5 |
| Nonane | 4.8 |
| 1-Nonanol | 3.3 |
| (Z)-6-nonen-1-ol | 2.3 |
| octan-2-one | 2.3 |
| octanol | 2.8 |
| 1-octen-3-ol | 2.7 |
| octyl acetate | 3.3 |
| octyl isobutyrate | 4.2 |
| oleic acid | 7.4 |
| 1-octyl-2-pyrrolidinone | 3.3 |
| Pentafluorobenzene | 3.0 |
| 2-phenyl ethyl octanoate | 4.7 |
| 2-phenylethyl 3-methyl-2-butenoate | 2.7 |
| 3-phenyl propanoic acid | 1.8 |
| 2-propenyl isothiocyanate | 1.2 |
| Pyridine | 0.8 |
| Tetradecane | 7.2 |
| Toluene | 2.5 |
| triacetin | 0.4 |
| 1,3,5-Trifluorobenzene | 2.6 |
| a,a,a-Trifluorotoluene | 3.6 |
| 1,3,5-trimethyl-Benzene (Mesitylene) | 3.6 |
| n-Undecane | 5.7 |
| undecan-2-one | 3.7 |
| Xylene | 3.1 |

The composition preferably contains a biocidally active compound in an amount effective to inhibit the growth of a pathogen on a surface to which the composition is applied.

In accordance with a further aspect of the present invention, there is provided a method for releasing a biocidally active compound from a composition comprising a biocidally active compound encapsulated within an adjuvant, wherein the adjuvant comprises a fungal cell or fragment thereof, the method comprising contacting the adjuvant with a surface of a microbe or a part thereof.

The surface of a microbe or a part thereof may comprise the cell wall, cell membrane, a biofilm, extracellular polysaccharide or proteinaceous matrix produced by the microbe.

In accordance with a further aspect of the present invention, there is provided a method for controlling a microbial infection comprising the use of a composition comprising a biocidally active compound encapsulated within an adjuvant, wherein the adjuvant comprises a fungal cell or fragment thereof, the method comprising contacting a surface of at least one microbe with the adjuvant.

In accordance with a further aspect of the present invention, there is provided the use of a composition comprising a biocidally active compound encapsulated within an adjuvant, wherein the adjuvant comprises a fungal cell or fragment thereof, for controlling a microbial infection.

Encapsulated compounds are described in WO 00/69440.

The fungal cell or a fragment thereof may be derived from one or more fungi from the group comprising Mastigomycotina, Zygoniycotina, Ascomycotina, Basidiomycotina and Deuteromycotina. Preferably, the fungal cell or a fragment thereof may be derived from one or more fungi from Ascomycotina. More preferably, the fungal cell or a fragment thereof may be derived from yeasts. More preferably still, the fungal cell or a fragment thereof may be derived from one or more of the group comprising Candida albicans, Blastomyces dermatitidis, Coccidioides immitis, Paracoccidioides brasiliensis, Penicillium marneffei and Saccharomyces cerevisiae. Even more preferably still, the fungal cell or a fragment thereof may be derived from Saccharomyces cerevisiae, such as common bakers yeast and yeast obtainable as a byproduct of ethanol biofule production.

In one composition according to the present invention, the fungal cell or fragment thereof is or is derived from yeast. More preferably, the yeast is or is derived from common bakers or ethanol biofuel yeast, or other Saccharomyces yeasts. When the adjuvant comprises a fungal cell, the fungal cell may be alive or dead. The adjuvant may comprise a plurality of fungal cells or fragments thereof, and may comprise a plurality of different types of fungal cells or fragments thereof. Cells suitable for use in the present invention may be the byproduct of the yeast extract process where a degree of cell contents have been removed and the cell membrane may be intact or damaged. Preferably cells will have intact cell walls and may be described as cell walls.

In an alternative embodiment the fungal cell may be derived from filamentous fungi. The fungal cell or fungal cell fragment is preferably derived from as Mucor and/or Rhizomucor, for high chitin cell wall and other species that are lower in chitin, such as Penicillium, Aspergillus and/or Fusarium. Preferably the fungal cell or fungal cell fragment may be derived from Saccharomyces cerevisiae, such as Bakers yeast, Williams yeast (obtainable from Aventine Renewable Energy Co., Inc. 1300 South 2^{nd} Street, Pekin, Ill., 61555-00, USA) or DCL blue label yeast obtainable from Lessafre at www.lesaffreyeastcorp.com.

The fungal cell or fungal cell fragment may be derived from yeast that is grown continually or grown in a batch. Yeast grown continually is usually used for the production of ethanol for fuel purposes and is adapted to a high alcohol environment. Such yeast is termed ethanol yeast or biofuel yeast of which Williams yeast is an example. Most preferably the fungal cell or fungal cell fragment is derived from biofuel yeast.

The microbial encapsulated product may be mixed with colourants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc. The fungal cell surface may also be dyed.

Methods of microbially encapsulating compounds are described in GB2162147, which describes special microbe cultivation methods to enhance microbial lipid content to a very high level whereby the encapsulating material is lipid soluble, and EP242135 which describes an improved method of encapsulation.

Preferably, the fungal cell is in grown form, i.e. It has been harvested from its culture medium, and is intact, i.e. not lysed. The fungal cell may be alive, may be a ghost cell or may be dead, i.e. unable to propagate.

In one composition according to the present invention, the fungal cell has an average diameter of more than 5 microns. The lipid content may be less than 60%, preferably less than 40%, more preferably less than 25%, still more preferably less than 15%, most preferably less than 5% by dry weight of the cell.

In accordance with a further aspect of the present invention there is provided a composition for use as a medicament, said composition comprising at least one essential oil and a fungal cell or fungal cell fragment wherein molecules of the at least one essential oil are encapsulated or partially encapsulated by the fungal cell or fungal cell fragment.

In one embodiment, there is provided a composition further comprising at least one essential oil encapsulated and/or non-encapsulated with the adjuvant.

In accordance with a further aspect of the present invention, there is provided a therapeutic formulation comprising a composition as described hereinabove. The formulation may comprise one or more excipients.

In accordance with a further aspect of the present invention there is provided the use of a composition for the manufacture of a medicament for the treatment or prophylaxis of microbial infection, the composition comprising at least one biocidally active compound and a fungal cell or fungal cell fragment, wherein molecules of the biocidally active compound are encapsulated or partially encapsulated by the fungal cell or fungal cell fragment.

The composition may be for the treatment of common spoilage fungi in plants (such as Fusarium sp., Penicillium sp., Aspergillus sp. Etc.), materials and food (fungi and other yeast), and medically important microbes (such as MRSA and Candida albicans). Preferably, the composition is for the treatment or prophylaxis of Staphylococcus, Candida albicans and/or Aspergillus niger infection. Strains of staphylococcus include S. aureus, S. epidermidis, S. saprophyticus, S. haemolyticus, Methicillin sensitive S. aureus (MSSA), Methicillin resistant S. aureus (MRSA) and Epidemic methicillin resistant S. aureus (EMRSA). More preferably, the composition is for the treatment of MRSA.

In accordance with a further aspect of the present invention there is provided a method of treating or preventing a microbial infection in a subject comprising administering to a subject a composition as described hereinabove.

The composition may be applied to the epidermis or epithelium exposed by a wound on a subject. The composition may alternatively be applied to a microbe surface.

The contaminated surface may comprise the epidermis of a human or animal, such as for example the scalp.

The composition may be applied to the surface of a substrate, such as for example a hospital bed, bed frame, floor, surgical instrument, devices for use in a hospital, mattress, bed sheets, clothing. The composition may be formulated in a mixture with a polymer. The composition may be dispersed throughout a polymer, providing the polymer with an integral anti-microbial agent. For example, plastics for use in manufacturing objects and/or devices which may come into contact with micro-organisms, such as, cutlery, surgical instruments, storage and/or transport containers, and in particular food storage and/or transport containers, work surfaces in kitchens, hospitals etc.

The composition may be dispersed within or applied at a surface of a sanitary towel, an ATB sanitiser, tissues, clothing, diaper etc.

Accordingly, the composition of the present invention may be formulated as a dry or liquid (emulsion or suspension) syrup, a sachet, a chewable, a chewing gum, an orodispersible, a dispersible effervescent, a dispersible tablet, a compressed buccal tablet, a compressed sublingual tablet, a chewable tablet, a melt-in-the-mouth, a lozenge, a paste, a powder, a gel, a tablet, a compressed sweet, a boiled sweet, a cream, a suppository, a snuff, a spray, an aerosol, a pessary, or an ointment. The composition may be formulated in a shampoo for treating and/or preventing dandruff.

In accordance with a further aspect of the present invention there is provided a method of manufacturing a composition as described hereinabove comprising contacting a capsule with the composition such that the composition is encapsulated by the capsule and retained passively

The present invention further provides a method of producing an encapsulated material comprising treating a grown intact microbe such as a fungus or bacterium by contiguous contact with an encapsulatable material in liquid form. The encapsulatable material being capable of diffusing into the microbial cell without causing total lysation thereof, and said treatment being carried out in the absence or presence of an organic lipid-extending substance (as defined in European Patent Specification No. 0085805) as solvent or microdispersant for the encapsulatable material and in the absence of a plasmolyser, whereby the material is absorbed by the microbe by diffusion across the microbial cell wall and is retained passively within the microbe (as described in European Patent Specification 0085805). The aforementioned prior methods rely either on special microbe cultivation conditions to enhance the microbial lipid content to a very high level or on the use of a lipid-extending substance, and the materials to be encapsulated must be either soluble in the microbial lipid or soluble or microdispersible in the lipid-extending substance, respectively.

In French Patent Specification No. 2179528 there is described a method of causing certain materials to be absorbed and/or fixed by microbes, in which a microbe such as pressed industrial yeast is treated with a plasmolyser, i.e. a substance which causes contraction or shrinking of the microbial cytoplasm by exosmosis of cytoplasmic fluid, and then an aqueous solution of a material such as neodymium chloride, magnesium chloride or onion juice is added under certain conditions so that the aqueous material is absorbed in place of the extracted cytoplasmic fluid

In one embodiment, the fungal cell is in grown form, i.e. it has been harvested from its culture medium, and is intact, i.e. not lysed. Suitably the microbe is alive, at least at the commencement of the treatment; however, a microbe which has been subjected to conditions (such as by irradiation of the microbe) to destroy its ability of propagate may be employed.

Preferably the capsule has a large size (cell size), for example of average diameter more than about 5 microns. Bacteria may have a smaller normal cell size of about 1 to 2 microns but may be cultivated to attain a larger size.

It is not necessary for the capsule to have a significant lipid content. Typically the lipid content may be not more than about 5%, for instance up to 3%, by dry weight of the microbe.

The encapsulatable material should be in liquid form during the treatment. It may be a liquid in its normal state, or it may be normally a solid in which case it should be dissolved or microdispersed in a solvent that is not miscible with the microbial lipid. Examples of suitable solvents are the lower alcohols such as methanol, ethanol and iso-propanol. The solvent may be removed after the encapsulation treatment, such as by spray-drying.

In one embodiment, the composition further comprises a carrier for co-encapsulation with biocide or essential oil.

In one embodiment, where the composition comprises an essential oil and a biocidal compound, the carrier comprises the essential oil.

The encapsulatable material need not be soluble in any lipid forming part of the capsule.

The method of encapsulation preferably comprises mixing the capsule with the composition in a liquid medium, especially an aqueous medium, to attain good dispersion and contact of the capsule with the composition. Accordingly, the composition may be mixed with an aqueous paste or slurry of the capsule, or the composition in a small quantity of water may be mixed with dry microbe. Preferably the composition forms an emulsion in the aqueous medium.

Encapsulation may be performed at normal ambient temperatures but preferably the temperature is elevated, at least during the initial stages, such as during at least the first 30 minutes, in order to expedite the encapsulation. A suitable elevated temperature may be in the range 35 to 70° C., more preferably 45-60° C.

The encapsulation may be observed microscopically as one or more globules of the composition inside the capsule. This may take a few hours.

In one embodiment, the capsule may be pretreated at an elevated temperature and/or with a proteolytic enzyme and/or with a chemical such as sodium hydroxide or a magnesium salt to enhance permeability prior to or in some cases during the encapsulation process. Such pretreatment may be carried out by incubating the microbe in water at an elevated temperature. The microbe may then be mixed with the material to be encapsulated at a lower temperature.

After encapsulation, the capsule may be treated to soften it in order to facilitate subsequent release of the encapsulated material, such as by treatment with a proteolytic enzyme or an alkali, or it may be treated to harden it in order to prevent premature liberation of the encapsulated material, such as by treatment with a dilute aqueous aldehyde solution. The encapsulated material may be released from the capsules when desired by, for instance, chemical, biodegradation or mechanical rupture of the microbial cell wall, and/or by subjecting the capsules to an environment in which the material diffuses gradually out through pores in the capsule and/or contacting the fungal cell wall or fragment thereof with epithelial cells and/or contacting the capsules with a compound that breaks down or disrupts the structure of cell membrane.

Capsules produced by the invention give rise to controlled release characteristics; for example when the release of the encapsulated material is delayed or prolonged by a slow or gradual rupture of the capsule or slow diffusion therefrom providing a sustained treatment.

In accordance with a further aspect of the present invention, there is provided an admixture of a plastics polymer and a composition as described hereinabove.

Specific embodiments of the present invention will now be described, by way of example only, with reference to the following figures and examples, in which:

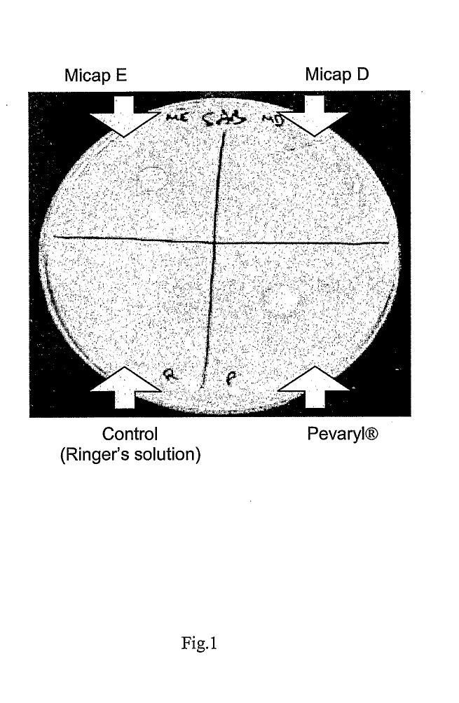

FIG. 1 illustrates the zones of inhibition demonstrating antifungal activity on a petri dish of various compositions;

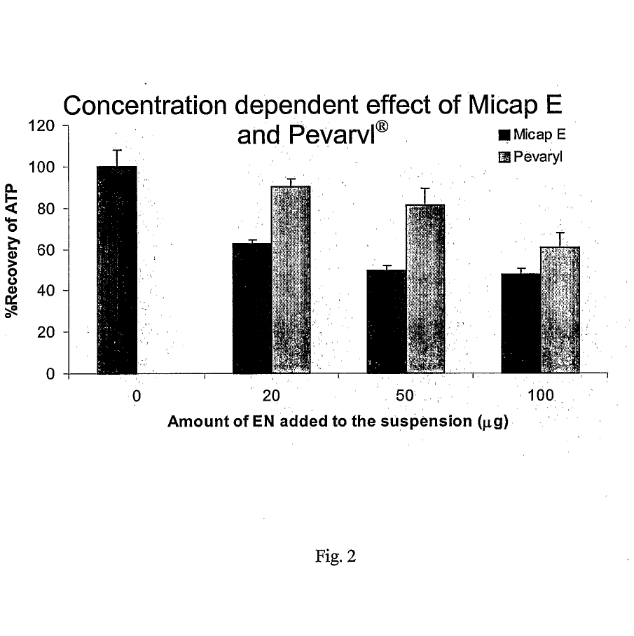

FIG. 2 illustrates the results of the concentration dependent effect of Micap E and Pevaryl® using the suspension method;

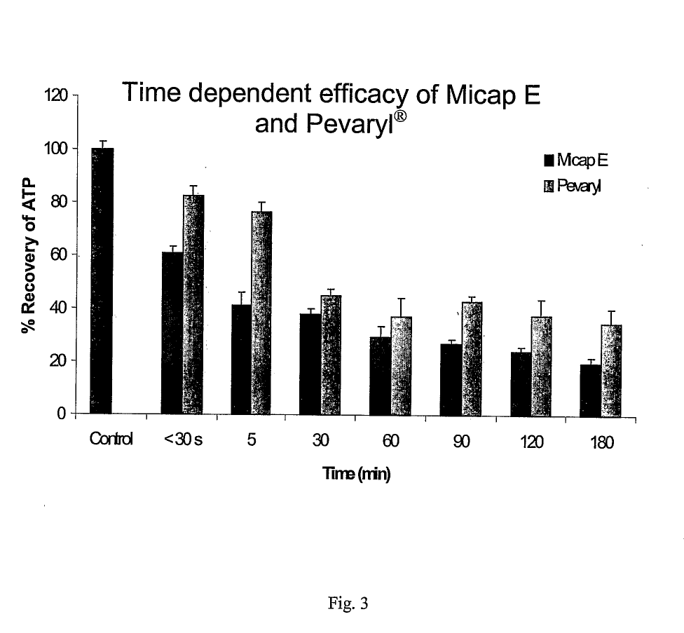

FIG. 3 illustrates the results of the time dependent efficacy of Micap E and Pevaryl® using the suspension method;

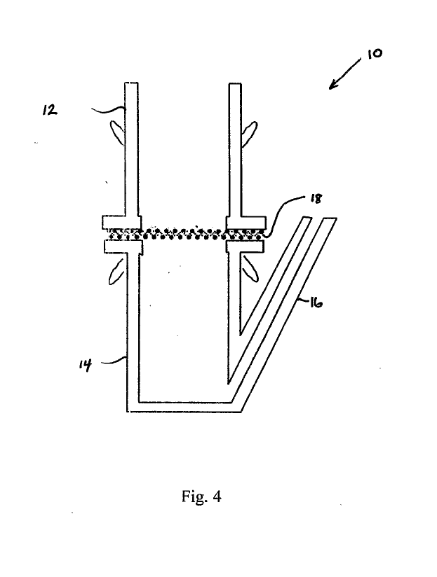

FIG. 4 illustrates a Franz cell for bioassay;

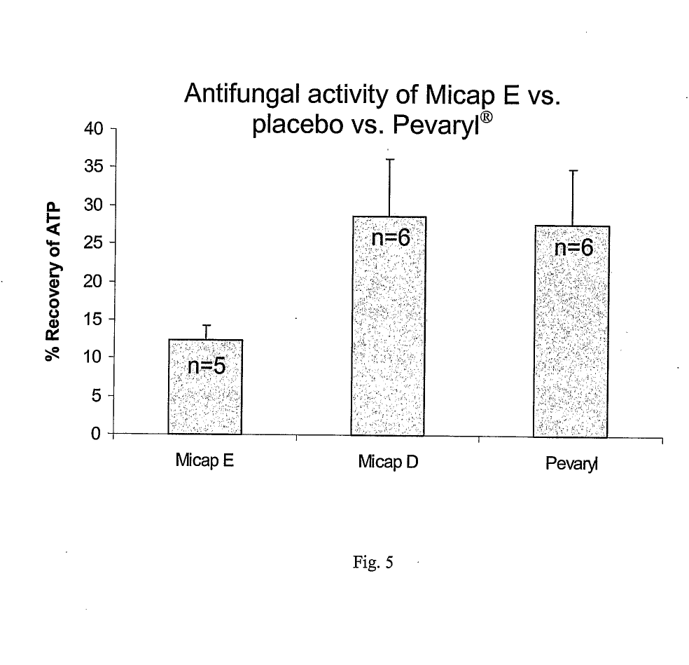

FIG. 5 illustrates the results of the antifungal activity of Micap E and Pevaryl®;

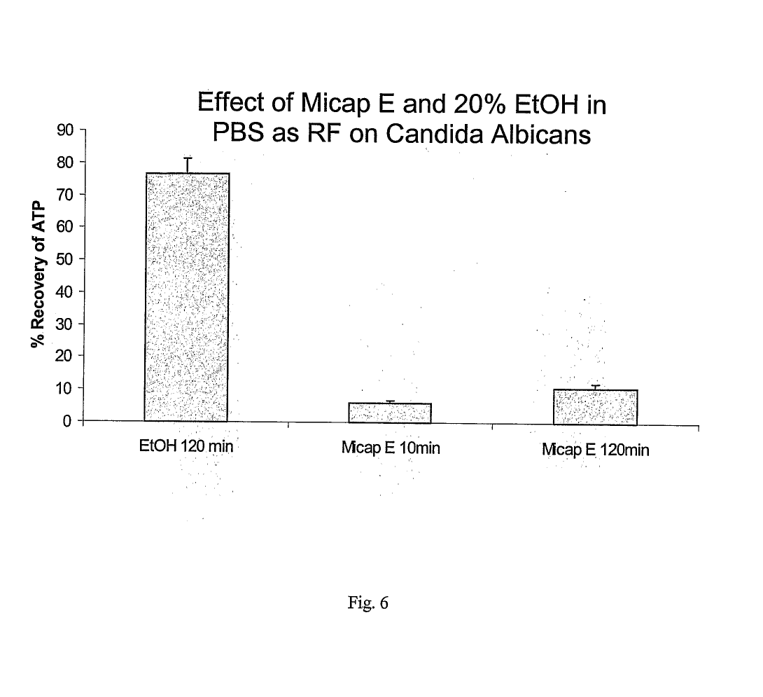

FIG. 6 illustrates the results of the effect of Micap E and 20% EtOH on cell viability; and

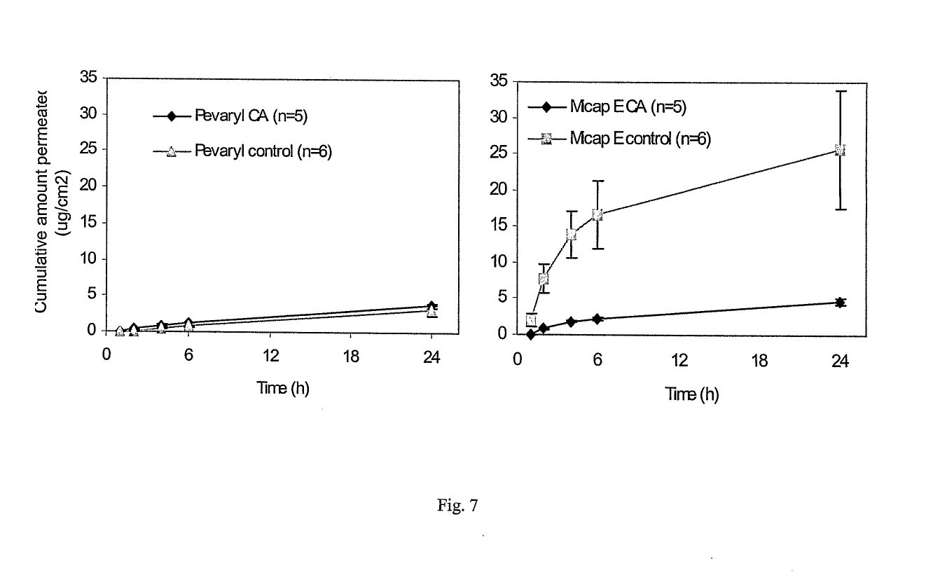

FIG. 7 illustrates the results of release of active on contact with a test organism.

### EXAMPLE 1

### Estimation of the Minimum Inhibitory Concentrations (MIC) of all Strains Against Triclosan (Direct Contact)

A stock solution of triclosan was prepared by adding 256 mg of triclosan to 10 ml of Dimethyl sulphoxide. A working solution was then prepared by diluting 1 ml of the stock solution in 9 ml of antibiotic assay broth (AAB). The working solution was then diluted from 2560 μg ml to 0.31 μg ml using AAB. Each dilution of triclosan (1 ml) was then vortex mixed with 19 ml of molten sensitivity test agar (STA) and poured into petri dishes. When set, the dilutions of triclosan in the STA ranged from 128 μg ml to 0.01 μg ml. An overnight broth culture (ONBC) of each bacterial strain was diluted 1/100 using AAB. Each strain was then placed onto the surface of the plates using a multi-point inoculator. Each plate was dried for 20 minutes and incubated for 24 hours at 37° C. The MIC of each strain was determined as the first plate in the dilution series showing no growth of the organism.

### Results

The MIC for triclosan against all strains of staphylococci.

| Strain | MIC (Mg ml⁻¹⁻) |
|---|---|
| Oxford S. aureus NCTC 6571 | 0.63 |
| S. aureus NCBC 11882 | 2 |
| S. epidermidis NCTC 11047 | 0.63 |
| S. epidermidis NCTC 7944 | 2 |
| S. saprophyticus NCIMB 8711 | 2 |
| S. haemolyticus NCTC 11042 | 1 |
| Strain T1 MSSA | 0.5 |
| Strain T4 MSSA | 0.25 |
| MSSA (4) | 0.5 |
| MSSA (46) | 2 |
| MSSA (47) | 0.5 |
| MSSA (48) | 1 |
| MRSA 11 | 0.5 |
| MRSA 12 | 0.5 |
| MRSA 13 | 2 |
| MRSA 14 | 0.5 |
| MRSA 15 | 1 |
| MRSA 16 | 0.062 |
| MRSA 17 | 0.25 |
| MRSA 20 | 1 |
| MRSA 26 | 2 |
| EMRSA m97 271 031 phage group 1 | 0.5 |
| EMRSA m97 271 038 phage group 2 | 0.5 |
| EMRSA j95 922 phage group 3 | 0.5 |
| EMRSA m97271052 phage group 4 | 0.062 |
| EMRSA m972 71041 phage group 5 | 0.062 |
| EMRSA m97 271 088 phage group 6 | 1 |
| EMRSA m97 271 047 phage group 8 | 2 |
| EMRSA m972 710 40 phage group 9 | 1 |
| EMRSA m97 271 032 phage group 10 | 0.5 |
| EMRSA m97 271 036 phage group 11 | 0.5 |
| EMRSA m97 271 042 phage group 12 | 1 |
| EMRSA m97 271 064 phage group 14 | 0.5 |
| EMRSA g96 139 515 phage group 15 | 2 |
| EMRSA g96 138 744 phage group 16 | 1 |
| EMRSA g96 136 210 phage group 17 | 1 |

The standard deviation for all was zero.

### Conclusion

The concentration of triclosan able to inhibit all strains of staphylococci was 2 μg ml⁻¹

### EXAMPLE 2

### Estimation of the Minimum Inhibitory Concentration (MIC) of Combined Triclosan and Essential Oils Against all Staphylococci Strains (Direct Contact)

Triclosan (4 μg ml) was added to 0.063% essential oil in equal volumes (concentrations of each were determined in previous experiments). This resulted in a ½ dilution of each component (2 μg ml triclosan and 0.031% essential oil). The combination was then double diluted and 1 ml of each dilution added to 19 ml of molten STA and dispensed into individual petri dishes. Each was allowed to set and dried for 30 minutes.

An overnight broth culture of each bacterial strain was then diluted 1/10 using AAB. Each strain was then placed onto the surface of the STA containing each combination of oil and triclosan using a multi-point inoculator. Each plate was allowed to dry for 20 minutes and incubated for 24 hours at 37° C. The MIC of each strain was determined as the first plate within the dilution series containing no growth of the organism

### Results

The MIC of combined essential oils and triclosan against all strains of staphylococci.

| | Manuka and | Manuka and | Manuka and | Tea tree and | Lemongrass and |
|---|---|---|---|---|---|
| Strain | Geranium (1) | Tea tree (2) | Lemongrass (3) | Lavender (4) | Geranium (5) |
| Oxford S. aureus NCTC 6571 | F | E | F | D | D |
| S. aureus NCBC 11882 | F | D | D | D | D |
| S. epidermidis NCTC 11047 | F | E | E | D | E |
| S. epidermidis NCTC 7944 | F | D | D | D | D |
| S. saprophyticus NCIMB 8711 | F | D | F | D | C |
| S. haemolyticus NCTC 11042 | F | D | F | D | E |
| Strain T1 MSSA | F | F | E | D | E |
| Strain T4 MSSA | F | E | E | D | E |
| MSSA (4) | F | E | F | D | E |
| MSSA (46) | F | D | D | F | D |
| MSSA (47) | F | F | F | D | E |
| MSSA (48) | F | E | E | F | E |
| MRSA 11 | F | D | D | D | E |
| MRSA 12 | F | E | E | D | E |
| MRSA 13 | F | D | D | F | C |
| MRSA 14 | F | F | E | D | E |
| MRSA 15 | F | F | E | D | E |
| MRSA 16 | F | F | E | D | E |
| MRSA 17 | F | F | D | D | E |
| MRSA 20 | F | F | F | F | E |
| MRSA 26 | F | D | D | D | E |
| EMRSA m97 271 031 phage | F | F | E | F | E |
| group 1 | | | | | |
| EMRSA m97 271 038 phage | F | E | F | D | E |
| group 2 | | | | | |
| EMRSA j95 922 phage group 3 | F | F | F | F | E |
| EMRSA m97271052 phage | F | F | E | F | E |
| group 4 | | | | | |
| EMRSA m972 71041 phage | F | F | F | F | E |
| group 5 | | | | | |
| EMRSA m97 271 088 phage | F | F | E | F | E |
| group 6 | | | | | |
| EMRSA m97 271 047 phage | F | E | E | D | E |
| group 8 | | | | | |
| EMRSA m972 710 40 phage | F | F | F | F | E |
| group 9 | | | | | |
| EMRSA m97 271 032 phage | F | E | E | F | E |
| group 10 | | | | | |
| EMRSA m97 271 036 phage | F | E | E | F | E |
| group 11 | | | | | |
| EMRSA m97 271 042 phage | F | E | E | F | E |
| group 12 | | | | | |
| EMRSA m97 271 064 phage | G | F | E | F | E |
| group 14 | | | | | |
| EMRSA g96 139 515 phage | F | D | D | D | D |
| group 15 | | | | | |
| EMRSA g96 138 744 phage | F | E | E | F | E |
| group 16 | | | | | |
| EMRSA g96 136 210 phage | F | E | E | F | E |
| group 17 | | | | | |

| | |
|---|---|
| Key for oils 1, 2, 3 | Key for oils 4 and 5 |

| | | | | | | | |
|---|---|---|---|---|---|---|---|
| | Oil | | Triclosan | | Oil | | Triclosan |
| A | 0.031 | + | 2 | A | 0.125 | + | 2 |
| B | 0.016 | + | 1 | B | 0.063 | + | 1 |
| C | 0.008 | + | 0.5 | C | 0.031 | + | 0.5 |
| D | 0.004 | + | 0.25 | D | 0.016 | + | 0.25 |
| E | 0.002 | + | 0.125 | E | 0.008 | + | 0.125 |
| F | 0.001 | + | 0.0625 | F | 0.004 | + | 0.0625 |
| G | 0.0005 | + | 0.03125 | G | 0.002 | + | 0.03125 |

### Conclusion

A lower concentration of combined triclosan and essential oils were more effective at inhibiting growth of all strains compared to when used singly.

### EXAMPLE 3

### Assessment of the Vapours of Triclosan Against all Strains of Staphylococcus (Vapour Phase)

A stock solution of triclosan was prepared by adding 256 mg of triclosan to 10 ml of Dimethyl sulphoxide. A working solution was then prepared by diluting 1 ml of the stock solution in 9 ml of antibiotic assay broth (AAB). The working solution (20 μl) was then placed onto 6 mm filter paper discs and placed into the lid of petri dishes.

An ONBC of each bacterial strain was diluted 1/100 and swabbed onto the surface of STA. The lids containing the discs were placed onto the petri dishes and the plates incubated for 24 hours at 37° C. The ZOI of each strain was determined by measuring the area of bacterial clearing (diameter, mm).

### Results

The ZOI of all strains against triclosan vapours

| Strain | 1 | 2 | Average | SD± |
|---|---|---|---|---|
| Oxford S. aureus NCTC 6571 | 50 | 52 | 51 | 1.41 |
| S. aureus NCBC 11882 | 47 | 49 | 48 | 1.41 |
| S. epidermidis NCTC 11047 | 50 | 51 | 50.5 | 0.71 |
| S. epidermidis NCTC 7944 | 50 | 51 | 50.5 | 0.71 |
| S. saprophyticus NCIMB 8711 | 33 | 42 | 37.5 | 6.36 |
| S. haemolyticus NCTC 11042 | 48 | 44 | 46 | 2.83 |
| Strain T1 MSSA | 46 | 45 | 45.5 | 0.71 |
| Strain T4 MSSA | 50 | 52 | 51 | 1.41 |
| MSSA (4) | 50 | 52 | 51 | 1.41 |
| MSSA (46) | 45 | 45 | 45 | 0.00 |
| MSSA (47) | 52 | 51 | 51.5 | 0.71 |
| MSSA (48) | 49 | 48 | 48.5 | 0.71 |
| MRSA 11 | 43 | 43 | 43 | 0.00 |
| MRSA 12 | 45 | 44 | 44.5 | 0.71 |
| MRSA 13 | 47 | 47 | 47 | 0.00 |
| MRSA 14 | 55 | 55 | 55 | 0.00 |
| MRSA 15 | 50 | 50 | 50 | 0.00 |
| MRSA 16 | 55 | 56 | 55.5 | 0.71 |
| MRSA 17 | 31 | 33 | 32 | 1.41 |
| MRSA 20 | 55 | 55 | 55 | 0.00 |
| MRSA 26 | 40 | 40 | 40 | 0.00 |
| EMRSA m97 271 031 phage group 1 | 42 | 40 | 41 | 1.41 |
| EMRSA m97 271 038 phage group 2 | 57 | 57 | 57 | 0.00 |
| EMRSA j95 922 phage group 3 | 55 | 53 | 54 | 1.41 |
| EMRSA m97271052 phage group 4 | 50 | 50 | 50 | 0 |
| EMRSA m972 71041 phage group 5 | 55 | 55 | 55 | 0 |
| EMRSA m97 271 088 phage group 6 | 52 | 52 | 52 | 0 |
| EMRSA m97 271 047 phage group 8 | 52 | 52 | 52 | 0 |
| EMRSA m972 710 40 phage group 9 | 55 | 55 | 55 | 0 |
| EMRSA m97 271 032 phage group 10 | 49 | 49 | 49 | 0 |
| EMRSA m97 271 036 phage group 11 | 52 | 52 | 52 | 0 |
| EMRSA m97 271 042 phage group 12 | 45 | 45 | 45 | 0 |
| EMRSA m97 271 064 phage group 14 | 35 | 35 | 35 | 0 |
| EMRSA g96 139 515 phage group 15 | 47 | 47 | 47 | 0 |
| EMRSA g96 138 744 phage group 16 | 50 | 50 | 50 | 0 |
| EMRSA g96 136 210 phage group 17 | 47 | 47 | 47 | 0 |

SD for all is zero

### Conclusion

The vapours of triclosan were effective at inhibiting growth of all staphylococcal strains.

### EXAMPLE 4

### Assessment of the Vapours of Triclosan and Essential Oils Against all Strains of Staphylococcus (Vapour Phase)

Triclosan (2560 μg ml⁻¹) was added in equal volumes to essential oil (100%) (The concentrations of essential oils were determined in previous experiments). This resulted in a ½ dilution of each component (1280 μg ml triclosan and 50% essential oil). The combination was then added to a 6 mm filter paper disc and placed on the lid of a petri dish.

An ONBC of each bacterial strain was diluted 1/100 and swabbed onto the surface of STA. The lids containing the discs were placed onto the petri dishes and the plates incubated for 24 hours at 37° C.

The STA of the petri dishes was then surface swabbed with a 1/100 dilution of the ONBC of 10 selected staphylococci strains and the petri dish placed onto the petri dish. Plates were incubated for 24 hours at 37° C. The ZOI of each strain was determined by measuring the area of bacterial clearing (diameter, mm).

Note: 10 strains were initially screened to assess if they had any effect on growth.

### Results

The ZOI of 10 staphylococcal strains against the vapours of
combined triclosan and essential oils.

| | | Lemongrass | Lemongrass |
|---|---|---|---|
| | Tea tree and | and | and |
| Strain | Lemongrass | Lavender | Geranium |
| Oxford S. aureus NCTC 6571 | FG | 19 | FG |
| S. aureus NCBC 11882 | 30 | FG | FG |
| S. epidermidis NCTC 11047 | 35 | FG | EG |
| S. haemolyticus NCTC 11042 | FG | FG | FG |
| Strain T4 MSSA | 15 | FG | FG |
| MRSA 12 | 12 | FG | 20 |
| MRSA 13 | 29 | FG | 10 |
| MRSA 14 | FG | FG | FG |
| EMRSA m97 271 064 | FG | FG | FG |
| phage group 14 | | | |
| EMRSA g96 139 | FG | FG | FG |
| 515 phage group 15 | | | |
| EMRSA g96 138 744 | FG | FG | FG |
| phage group 16 | | | |

FG = Full growth (no area of clearing)
Standard deviations for each were zero.

### Conclusion

When essential oils and triclosan were combined, the effect of the vapours on the ZOI was less effective than when used singly. This indicates that when combined an antagonistic effect between the triclosan and oils were occurring.

### EXAMPLE 5

### Estimation of the Minimum Inhibitory Concentrations (MIC) of Essential Oils Against Gram Negative Organisms

### Methods

Selected Bacterial Strains

Enterococcus faecium (n=3), Staphylococcus aureus (n=6), Staphylococcus saprophyticus (n=1), Citrobacter sp. (n=2), Klebsiella sp. (n=2), E. coli (n=2), Acinetobacter sp. (n=2) and Pseudonoman aeruginosa (n=1) (See appendix). Strains 1-10 were Gram positive organisms and strains 11-20 were Gram negative organisms.

Selected Essential Oils

Geranium (Egypt) (Pelargonium graveolens), Lemongrass (East Indian) (Cymbopogon flexuosus), Rosewood (Aniba rosaeodora) (Essentially oils, Churchill, Oxon).

Preparation of Essential Oil Combinations

Combinations of essential oils were performed in ratios of 50:50, 75:25 and 33:33:33

Example of an Essential Oil Combination: 75:25 Lemongrass and Rosewood
1600 μl of essential oil (1200 μl Lemongrass: 400 μl Rosewood)+400 μl of AAB (antibiotic assay broth)=2 ml of 80% essential oil stock mixture
1 ml of 80% stock mixture was used to create doubling dilutions and 1 ml was added to 19 ml of diagnostic sensitivity test agar (DST). This gave a final dilution of 4% essential oil mixture within the DST agar.

Estimation of the Minimum Inhibitory Concentrations (MIC) of all Strains Against Single and Combined Essential Oils (Direct Contact)

Each individual essential oil and essential oil combination was diluted from 80% to 0.062% using antibiotic assay broth (AAB). Each dilution (1 ml) was then vortex mixed with 19 ml of molten diagnostic sensitivity test agar (DST at 40° C.) containing 0.5% Tween, and dispensed into individual Petri dishes. Plates were allowed to set and dried for 30 minutes. After addition of the dilutions to the DST, each essential oil or essential oil combination resulted in a dilution of 4% to 0.031%.

An overnight broth culture (ONBC) of each bacterial strain was diluted 1/100 (Approx. 10⁵ cfu/ml) using AAB. Each strain was then placed onto the surface of the DST agar containing the essential oil and essential oil combinations, using a multi-point inoculator. Plates were dried for 20 minutes and incubated for 24 hours at 37° C. The minimum inhibitory concentration (MIC) of each oil/oil combination was determined as the first plate within the dilution series showing no growth of the organism.
Controls were carried out: this involved growth of the organisms on agar only (containing 0.5% Tween) and on agar containing antibiotic assay broth only.

### Results

When used individually, lemongrass and geranium were the most effective at inhibiting growth of Gram +ve organisms, whereas rosewood was the least effective. However, rosewood was the most effective oil at inhibiting growth of Gram −ve organisms, whereas geranium was highly ineffective (see table 3).
When oils were used in ratios of 50:50, a combination of lemongrass and geranium was the most effective at inhibiting growth of Gram +ve organisms, and a combination of geranium and rosewood was the least effective. A combination of lemongrass and rosewood was most effective at inhibiting growth of Gram −ve organisms and lemongrass and geranium was the least effective (see table 4).
When oils were used in ratios of 75:25, a combination of geranium and lemongrass and lemongrass and geranium were the most effective at inhibiting growth of Gram +ve organisms, and a combination of rosewood and geranium were the least effective. A combination of rosewood and lemongrass were the most effective at inhibiting growth of Gram −ve organisms and a combination of geranium and lemongrass and geranium and rosewood were the least effective (see table 5).
When oils were used in a ratio of 33:33:33: the combination was more effective against Gram +ve organisms than Gram −ve organisms (see table 6).
Bacterial strains showing to be the most resistant against the essential oils and combinations were Pseudomonas aeruginosa and Escherichia coli (NCIMB 9484)

TABLE 3
MIC of Lemongrass, Geranium and Rosewood against fecal and urogenital bacteria.

| | | Lemongrass | Geranium | Rosewood |
|---|---|---|---|---|
| Bacteria | | (MIC %) | (MIC %) | (MIC %) |
| 1 | Enterococcus faecium 3 | 0.125 | 0.125 | 0.25 |
| 2 | Enterococcus faecium 4 | 0.125 | 0.125 | 0.25 |
| 3 | Enterococcus faecalis (NCTC 775) | 0.125 | 0.125 | 0.25 |
| 4 | Staphylococcus aureus MRSA 12 | 0.125 | 0.125 | 0.25 |
| 5 | Staphylococcus aureus MRSA 13 | 0.125 | 0.125 | 0.25 |
| 6 | Staphylococcus aureus MRSA 15 | 0.125 | 0.125 | 0.25 |
| 7 | Staphylococcus aureus MRSA 16 | 0.06 | 0.125 | 0.25 |
| 8 | Staphylococcus aureus (Oxford Strain) NCTC 6571 | 0.06 | 0.125 | 0.25 |
| 9 | Staphylococcus saprophyticus (8771) | 0.125 | 0.125 | 0.25 |
| 10 | Staphylococcus aureus MRSA 26 | 0.125 | 0.125 | 0.25 |
| 11 | Citrobacter freundii (82073) | 0.5 | >4 | 0.25 |
| 12 | Citrobacter sp (61395) | 0.5 | >4 | 0.25 |
| 13 | Klebsiella pueumoniae (6655) | 1 | >4 | 0.25 |
| 14 | Klebsiella oxytoca (6653) | 0.25 | >4 | 0.25 |
| 15 | Escherichia coli (NCTC 9001) | 0.5 | >4 | 0.25 |
| 16 | Acinetobacter junii (A99) | 0.25 | >4 | 0.25 |
| 17 | Acinetobacter baumanii (H100) | 0.5 | 0.5 | 0.25 |
| 18 | Acinetobacter baumanii (A483) | 0.5 | 0.5 | 0.25 |
| 19 | Pseudomonas aeruginosa (NCTC 6749) | 1 | >4 | >4 |
| 20 | Escherichia coli (NCIMB 9484) | 0.25 | 0.5 | >4 |

TABLE 4
MIC of Lemongrass, Geranium and Rosewood combinations against fecal
and urogenital bacteria.

| | | 50 | 50 | 50 |
|---|---|---|---|---|
| | | Geranium:50 | Lemongrass:50 | Lemongrass:50 |
| | | Rosewood | Geranium | Rosewood |
| Bacteria | | (MIC %) | (MIC %) | (MIC %) |
| 1 | Enterococcus faecium 3 | 0.25 | 0.125 | 0.25 |
| 2 | Enterococcus faecium 4 | 0.25 | 0.125 | 0.25 |
| 3 | Enterococcus faecalis (NCTC 775) | 0.5 | 0.125 | 0.25 |
| 4 | Staphylococcus aureus MRSA 12 | 0.5 | 0.125 | 0.25 |
| 5 | Staphylococcus aureus MRSA 13 | 0.5 | 0.125 | 0.25 |
| 6 | Staphylococcus aureus MRSA 15 | 0.25 | 0.125 | 0.25 |
| 7 | Staphylococcus aureus MRSA 16 | 0.25 | 0.125 | 0.25 |
| 8 | Staphylococcus aureus (Oxford Strain) NCTC 6571 | 0.25 | 0.125 | 0.25 |
| 9 | Staphylococcus saprophyticus (8771) | 0.25 | 0.125 | 0.25 |
| 10 | Staphylococcus aureus MRSA 26 | 0.25 | 0.125 | 0.25 |
| 11 | Citrobacter freundii (82073) | 0.5 | 1 | 0.25 |
| 12 | Citrobacter sp (61395) | 1 | 1 | 0.25 |
| 13 | Klebsiella pneumoniae (6655) | 2 | >4 | 0.25 |
| 14 | Klebsiella oxytoca (6653) | 0.5 | 1 | 0.25 |
| 15 | Escherichia coli (NCTC 9001) | 1 | 2 | 0.25 |
| 16 | Acinetobacter junii (A99) | 0.125 | 2 | 0.25 |
| 17 | Acinetobacter baumanii (H100) | 1 | 1 | 0.25 |
| 18 | Acinetobacter baumanii (A483) | 1 | 1 | 0.25 |
| 19 | Pseudomonas aeruginosa (NCTC 6749) | 4 | >4 | >4 |
| 20 | Escherichia coli (NCIMB 9484) | 0.25 | >4 | >4 |

TABLE 5
MIC of Lemongrass, Geranium and Rosewood combinations against fecal
and urogenital bacteria.

| | | 75 | 75 | 75 | 75 | 75 | 75 |
|---|---|---|---|---|---|---|---|
| | | Rosewood:25 | Geranium:25 | Lemongrass:25 | Rosewood:25 | lemongrass:25 | Geranium:25 |
| | | Geranium | Lemongrass | Geranium | Lemongrass | rosewood | Rosewood |
| Bacteria | | (MIC %) | (MIC %) | (MIC %) | (MIC %) | (MIC %) | (MIC %) |
| 1 | Enterococcus faecium 3 | 0.5 | 0.125 | 0.125 | 0.25 | 0.25 | 0.25 |
| 2 | Enterococcus faecium 4 | 0.5 | 0.125 | 0.125 | 0.25 | 0.25 | 0.25 |
| 3 | Enterococcus faecalis | 0.5 | 0.25 | 0.25 | 0.25 | 0.25 | 0.25 |
| | (NCTC 775) | | | | | | |
| 4 | S. aureus MRSA 12 | 0.5 | 0.125 | 0.125 | 0.25 | 0.125 | 0.125 |
| 5 | S. aureus MRSA 13 | 0.25 | 0.125 | 0.125 | 0.25 | 0.125 | 0.125 |
| 6 | S. aureus MRSA 15 | 0.125 | 0.125 | 0.125 | 0.25 | 0.125 | 0.125 |
| 7 | S. aureus MRSA 16 | 0.125 | 0.125 | 0.125 | 0.25 | 0.125 | 0.125 |
| 8 | S. aureus (Oxford) NCTC 6571 | 0.125 | 0.125 | 0.125 | 0.25 | 0.125 | 0.125 |
| 9 | S. saprophyticus (8771) | 0.25 | 0.125 | 0.125 | 0.25 | 0.125 | 0.125 |
| 10 | S. MRSA 26 | 0.125 | 0.125 | 0.125 | 0.25 | 0.125 | 0.125 |
| 11 | Citrobacter freundii (82073) | 0.25 | 4 | 0.25 | 0.25 | 0.5 | 4 |
| 12 | Citrobacter sp (61395) | 0.25 | >4 | 0.125 | 0.25 | 1 | 2 |
| 13 | Klebsiella pneumoniae (6655) | 0.25 | >4 | 0.25 | 0.25 | 1 | >4 |
| 14 | Klebsiella oxytoca (6653) | 0.125 | 4 | 0.125 | 0.25 | 0.5 | 1 |
| 15 | Escherichia coli (NCTC 9001) | 0.25 | >4 | 0.5 | 0.25 | 0.5 | >4 |
| 16 | Acinetobacter junii (A99) | 1 | >4 | 0.25 | 0.25 | 1 | 4 |
| 17 | Acinetobacter baumanii (H100) | 0.25 | >4 | 0.25 | 0.25 | 0.5 | 4 |
| 18 | Acinetobacter baumanii (A483) | 0.25 | >4 | 0.5 | 0.25 | 0.5 | 4 |
| 19 | Pseudomonas aeruginosa | >4 | >4 | >4 | >4 | >4 | >4 |
| | (NCTC 6749) | | | | | | |
| 20 | Escherichia coli (NCIMB 9484) | >4 | >4 | >4 | >4 | 2 | >4 |

TABLE 6
MIC of Lemongrass, Geranium and Rosewood combinations
against fecal and urogenital bacteria.

| | | 33 lemongrass:33 |
|---|---|---|
| | | rosewood:33 |
| Bacteria | | geranium (MIC %) |
| 1 | Enterococcus faecium 3 | 0.125 |
| 2 | Enterococcus faecium 4 | 0.125 |
| 3 | Enterococcus faecalis (NCTC 775) | 0.125 |
| 4 | Staphylococcus aureus MRSA 12 | 0.125 |
| 5 | Staphylococcus aureus MRSA 13 | 0.125 |
| 6 | Staphylococcus aureus MRSA 15 | 0.125 |
| 7 | Staphylococcus aureus MRSA 16 | 0.125 |
| 8 | Staphylococcus aureus | 0.125 |
| | (Oxford Strain) NCTC 6571 | |
| 9 | Staphylococcus saprophyticus (8771) | 0.125 |
| 10 | Staphylococcus aureus MRSA 26 | 0.125 |
| 11 | Citrobacter freundii (82073) | 0.5 |
| 12 | Citrobacter sp (61395) | 0.25 |
| 13 | Klebsiella pneumoniae (6655) | 0.5 |
| 14 | Klebsiella oxytoca (6653) | 0.25 |
| 15 | Escherichia coli (NCTC 9001) | 0.5 |
| 16 | Acinetobacter junii (A99) | 0.5 |
| 17 | Acinetobacter baumanii (H100) | 0.25 |
| 18 | Acinetobacter baumanii (A483) | 0.25 |
| 19 | Pseudomonas aeruginosa (NCTC 6749) | >4 |
| 20 | Escherichia coli (NCIMB 9484) | >4 |

Essential oils and encapsulated essential oils can be used to reduce (and possibly kill) the bacteria found in urinal and faecal contamination of surfaces, but further tests need to be carried out to determine this in situ.
Lemongrass and rosewood oil is effective against both Gram +ve and Gram −ve organisms in a ratio of 50:50. These oils created large zones of inhibition against the same organisms.

### EXAMPLE 6

### Biocide Encapsulations

### Encapsulation of Biocidally Active Compounds

### Apparatus

### Overhead Stirrer—typically Stuart Scientific SS20

### Paddle stirring rod

### Temperature controlled waterbath

### Reaction Vessel

### Benchtop centrifuge

### Buchi Lab Spray Dryer model B290

### Magnetic stirrer plate

### Magnetic Flea

### Beaker

### Method

To prepare the yeast slurry, water was weighed into the reaction vessel and the water was warmed in a water bath. The requisite quantity of yeast powder was added slowly with stirring to create a well dispersed suspension and the yeast was fully hydrated. Heating of the yeast suspension continued until the desired encapsulation temperature was reached. Liquid biocides such as n-Octyl isothiazolinone were added directly to the yeast suspension and thoroughly mixed. For biocides that are solid at standard conditions a carrier solvent was used, such as benzyl alcohol, to dissolve the biocide in prior to addition to the yeast slurry. The carrier solvent may require heating to the encapsulation temperature prior to addition of the solid biocide in order to increase the solubility of the biocide in the carrier.

A stirring rod was fitted into the reaction vessel and the unit was placed in a temperature controlled water bath and connected to an overhead stirrer. The mixture was stirred for 16 hours at 40° C. before separation of the yeast encapsulated active from the liquid suspension. Separation was achieved by centrifuging the yeast dispersion in a bench top centrifuge at 3200 rpm for 20 minutes. The supernatant was discarded and the pellet re-suspended in water to create a feed stock with a solids content of approximately 20%, suitable for spray drying. The sample was placed in a beaker on a magnetic stirrer and the yeast capsules were spray dried on a Buchi lab spray dryer model B290.

### Terbutryn

15 g Terbutryn
75 g Benzyl alcohol
180 g Dried Yeast
380 g water

### Mix at 40 C

### IPBC

12.5 g IPBC
12.5 g Benzyl alcohol
50 g yeast dry weight
140 g water

### Mix at 40 C

### Menthol

17.5 g Menthol
17.5 g Benzyl alcohol
75 g yeast dry weight
180 g water

### Mix at 40 C

### Econazole Nitrate

### Econazole Nitrate 25% active dissolved in carrier

### Benzyl Alcohol

### Yeast

### Water

### Mix at 60 C

### Tebuconazole

30 g Tebuconazole
100 g Benzyl Alcohol
260 g Yeast
610 g water

### Mix at 40 C

### N-butyl-1,2-benzisothiazolin-3-one (BBIT)

25 g N-butyl-1,2-benzisothiazolin-3-one (BBIT)
50 g Yeast
110 g Water

### Mix at 40 C

### Octyl Isothiazolinone

25 g octyl isothiazolinone
50 g Yeast
170 g water

### Mix at 40 C

### Biocidally Active Compound Release and Antimicrobial Performance

Candida Albicans was chosen as the indicator organism, which is a common organism detected in many infections.

The concentration of organisms in suspension was determined by fluorescence technique and a viable count used for confirmation by conducting an ATP assay where ATP in cell lysate represents a measure for viable cells. Therefore, the number of viable cells after exposure to drug can be measured for drug activity and was used for suspension method or skin model.

The antifungal activity of Micap formulations using Candida Albicans as the indicator organism was determined in the three chosen models:

1. zones of inhibition (agar plate)
2. suspension method with ATP assay

### 3. In-vitro fungal infection of human skin in a Franz cell set up with ATP assay

For comparison, a commercial product Pevaryl® was subjected to the same procedures.

The following compositions were prepared:

| | |
|---|---|
| Econazole nitrate encapsulated in biofuel yeast | Micap A |
| Benzyl alcohol encapsulated in biofuel yeast | Micap B |
| (placebo for Micap A) | |
| Washed biofuel yeast | Micap C |
| Econazole nitrate encapsulated medical yeast | Micap E |
| Benzyl alcohol encapsulated in medical yeast | Micap D |
| (placebo for Micap E) | |
| Medical yeast | Micap F |
| Benzyl alcohol | BA |
| Econazole nitrate | EN |
| 2,4 Dichloroacetophone | DCAP |

### EXAMPLE 7

### Results: Zone of Inhibition

The biocidal activity of Micap E and D was determined by measuring zones of inhibition on agar plates which had been cultured with Candida Albicans. Sensitivity discs carrying Micap E, Micap D, Pevaryl® and a control, Ringer's solution were deposited on the Candida Albicans infected agar and incubated overnight. The zones of inhibition were subsequently measured.

As can be clearly seen in FIG. 1, Micap E shows a larger zone of inhibition than Pevaryl®, whilst no effect was observed from the control or placebo, Micap D.

### EXAMPLE 8

### Results: Suspension Method

Samples of Micap E and Pevaryl® having varied EN concentrations 20, 50 and 100 μg were incubated for 30 minutes with Candida Albicans in water (5×10⁷) followed by cell lysis and ATP assay to determine cell viability.

FIG. 2, clearly demonstrates that Micap E and Pevaryl® showed significant antifungal activity, whilst Micap E was more effective than Pevaryl®.

When samples of Micap E and Pevaryl® having 200 μg EN with Candida Albicans in water (8×10⁷) and the incubation time was varied between less than 30 seconds to 180 minutes, as shown in FIG. 3, Micap E was consistently more effective than Pevaryl®.

### EXAMPLE 9

### Franz Cell Studies

Stratum corneum (SC) sheets were infected with microorganisms in-vitro and a Franz cell was set-up to provide more realistic conditions than agar plate test

As shown in FIG. 4, Franz cell 10 has an upper donor well 12, a receptor well 14 and a sampling side arm 16 has a stratum corneum sheet 18 mounted between upper donor well 12 and receptor well 14.

A sample of Candida albicans (5×10⁷) was dried on the stratum corneum sheet and PBS used as receiver fluid. The Micap formulations were used at 10% aqueous suspensions and the Micap formulations and Pevaryl® were applied to the stratum corneum sheet and incubated at 37° C. The reaction was stopped after set time by dismantling the Franz cell and transferring the stratum corneum into TCA. ATP assay was then carried out on the cell lysate to determine activity.

### EXAMPLE 10

### Results: Franz Cell Bioassay

FIG. 5 shows the percentage recovery of ATP following 10 minutes incubation of Candida albicans with Micap E, Micap D and Pevaryl® where the concentration of econazole is 250 μg per cell. Micap E is clearly more effective than the placebo, Micap D, and Pevaryl®. A comparison of Micap E with 20% Ethanol, as shown in FIG. 6, demonstrates that after 2 hours incubation, 20% ethanol reduces ATP to 75%, whilst Micap E in a period of 10 minutes showed total kill of the organism.

### EXAMPLE 11

### Release of Active

A Franz cell as shown if FIG. 4 was used to demonstrate the release of the active from a microbial microcapsule on contact with a target organism. The receiver fluid in the receptor well was 20% EtOH in PBS. The concentration of EN per cell was 250 μg. 25 μl 10% Micap E in water and ˜25 mg Pevaryl® were incubated at 37° C. and the receiver fluid sampled over 72 hours (at 1, 2, 4, 6, 24, 48 and 72 h)

FIG. 7 shows the results of the example and clearly demonstrates that when Micap E is in contact with the target organism, Micap E releases much more active than in the absence of the target organism.

### REFERENCES

1. Lawrence J C. Burn Bacteriology during the last 50 years. Burns 1992; 18: (suppl 2), 23-29
2. Childs, C., Edwards-Jones, V., Heathcote, D. M., Dawson, M. & Davenport, P Patterns of Staphylococcus aureus colonization, toxin production, immunity and illness in burned children Bruns (1994) 20, 514-521.
3. Voss A. Milatovic D. Wallrauch-Schwarz C. Rosdahl V T. Braveny I, Methicillin-Resistant Staphylococcus Aureus in Europe. Eur J Clin Microbiol Infect Dis: 1994, 13: 50-55.
4. Walker J, Borrow R, Edwards-Jones V, Oppenheim B A, Fox A J. Epidemiological characterization of methicillin-resistant Staphylococcus aureus isolated in the North West of England by protein A (spa) and coagulase (coa) gene polymorphisms. Epidemiol. Infect. 1998. 121: 507-514.
5. Anon., 1998 Methicillin-resistant Staogtkiciccys ayreys, Commun Dis Rep Weekly: 8: 372.
6. Smith T L. Pearson M C, Wilcox R. Cruz C. Lancaster M V. Robinson-Dunn B. Tenover F C. Zervos M J. B and J D White E. Jarvis J D. Emergence of vancomycin resistance in Staphylococcus aureus. Glycopeptide-Intermediate Staphylococcus aureus Working Group. New. Engl. Med. 1999, 240: 493-501.
7. Lawrence J C. Dressings and wound Infection. America Journal of Surgery 1994; 167: (suppl 1A), 21S-24S.
8. Edwards-Jones, V. Dawson, M M, & Childs, C A survey into TSS in UK Burns Units Burns, 2000.
9. Herruzo-Cabrera, R. Vizcaino-Alcaide, M. J. Mayer, R. F. & Rey-Calero, J. A new in vitro model to test the effectiveness of topical antimicrobial agents. Use of an artificial eschar. Burns, 1992; 18; 35-38.

1. A composition comprising at least one biocidally active compound encapsulated within an adjuvant, wherein the adjuvant comprises a fungal cell or fragment thereof.

2. A composition as claimed in claim 1 wherein, the fragment of fungal cell comprises a fungal cell wall or a part thereof.

3. A composition as claimed in claim 1 wherein the biocidally active compound is lipophilic or comprises a lipophilic moiety.

4. A composition as claimed in claim 3 wherein the biocidally active compound is substantially lipophilic.

5. A composition as claimed in claim 3 wherein the biocidally active compound is derived from a lipophobic compound and chemically modified to be substantially lipophilic.

6. A composition as claimed in claim 1 wherein the biocidally active compound has a positive partition coefficient (LogP_{o/w}).

7. A composition as claimed in claim 6 wherein the biocidally active compound has a positive partition coefficient (LogP_{o/w}) greater than 0.1.

8. A composition as claimed in claim 7 wherein the biocidally active compound has a positive partition coefficient (LogP_{o/w}) in the range 0.1-10

9. A composition as claimed in claim 8 wherein the biocidally active compound has a positive partition coefficient (LogP_{o/w}) in the range 0.5-10

10. A composition as claimed in claim 9 wherein the biocidally active compound has a positive partition coefficient (LogP_{o/w}) in the range 2.0-7.0.

11. A composition as claimed in claim 1 wherein the biocidally active compound has a pH in the range pH1.0-12.0.

12. (canceled)

13. A composition as claimed in claim 11 wherein the biocidally active compound has a pH in the range pH4-9.

14. A composition as claimed in claim 1 wherein the biocidally active compound is acid and has a pKa between 2.0-7.0.

15. A composition as claimed in claim 1 wherein the biocidally active compound is basic and has a pKa between 7.0-12.

16. A composition as claimed in claim 1 wherein the biocidally active compound is present in an amount from 1-50 g/100 g of composition.

17. A composition as claimed in claim 1 wherein the biocidally active compound is a liquid at s.t.p or soluble in an organic solvent.

18. A composition as claimed in claim 17 wherein the biocidally active compound is soluble in the solvent at a level above 10 g/l.

19. A composition as claimed in claim 1 further comprising a carrier that facilitates encapsulation of the biocidally active compound within the adjuvant.

20. A composition as claimed in claim 19 wherein the carrier is selected from one or more compounds selected from the group consisting of: alkanes, alkenes, alkynes, aldehydes, ketones, monocyclics, polycyclics, heterocyclics, monoterpenes, furans, pyroles, pyrazines, azoles, carboxylic acids, benzenes, alkyl halides, alcohols, ethers, epoxides, esters, fatty acids and essential oils.

21. A composition as claimed in claim 19 wherein the carrier comprises one or more of the compounds listed in Table 1.

22. A composition as claimed in claim 19 wherein the carrier has a molecular weight in the range of 100-700.

23. A composition as claimed in claim 1 wherein the fungal cell or a fragment thereof is derived from one or more fungi selected from the group consisting of Mastigomycotina, Zygomycotina, Ascomycotina, Basidiomycotina and Deuteromycotina.

24. A composition as claimed in claim 23 wherein the fungal cell or fragment thereof is derived from one or more fungi from Ascomycotina.

25. A composition as claimed in claim 1 wherein the fungal cell or a fragment thereof is derived from yeast.

26. A composition as claimed in claim 25 wherein the yeast is selected from one or more of the group consisting of Candida albicans, Blastomyces dermatitidis, Coccidioides immitis, Paracoccidioides brasiliensis, Penicillium marneffei and Saccharomyces cerevisiae.

27. A composition as claimed in claim 26 wherein the yeast is Saccharomyces cerevisiae.

28. A composition as claimed in claim 25 wherein the fungal cell or fragment thereof is derived from a biofuel yeast.

29. A composition as claimed in claim 1 wherein the adjuvant comprises a fungal cell which is alive or dead.

30. A composition as claimed in claim 1 wherein the composition is formulated into any one of: solutions, emulsions, suspensions, powders, foams, pastes, granules, aerosols, active-compound-impregnated natural and/or synthetic materials, polymeric substances, coating compositions for seed, formulations with smokes, fumigating cartridges, fumigating cans, fumigating coils, and also ULV cold mist and warm mist formulations.

31. A composition as claimed in claim 1 wherein the biocidally active compound is a fungicide and/or a bactericide.

32. A composition as claimed in claim 31 wherein the biocidally active compound is an antibiotic.

33. A composition as claimed in claim 32 wherein the biocidally active compound comprises mupirocin, fucoidin and/or gentamicin.

34. A composition as claimed in claim 31 wherein the biocidally active compound is selected from one or more compounds selected from the group consisting of: phenols, cresols, acids, esters, alkalis, chlorine release agents, iodine compounds, quaternary ammonium compounds, biguanides, diamidines, aldehydes, alcohols, heavy metal derivatives, vapour phase disinfectants, sulphates and nitrites.

35. A composition as claimed in claim 1 wherein the biocidally active compound comprises one or more essential oils.

36. A composition as claimed in claim 1 wherein the biocidally active compound is present in an amount effective to inhibit the growth of a pathogen.

37. A composition as claimed in claim 1 wherein the biocidally active compound comprises econazole, triclosan, chlorhexidine, povidone iodine and/or silver sulphadiazine, terbutryn, IPBC, menthol, econazole tebuconazole, N-butyl-1,2-benzisothiazolin-3-one (BBIT) and/or octyl isothiazolinone.

38. A method for releasing a biocidally active compound from the composition of claim 1 comprising contacting the adjuvant with a surface of a microbe or a part thereof.

39. A method as claimed in claim 38 wherein the surface of a microbe comprises the cell wall or the cell membrane, extracellular polysaccharide or proteinaceous matrix produced by the microbe

40. A method for controlling a microbial infection comprising contacting a surface of at least one microbe with the adjuvant contained in the composition of claim 1.

41. (canceled)

42. A therapeutic formulation comprising a composition as claimed in claim 1.

43. (canceled)

44. A method of treating or preventing a microbial infection in a subject comprising administering to a subject a composition as claimed in claim 1.

45. An admixture comprising a plastics polymer and a composition as claimed in claim 1.

46. A sanitary towel comprising a composition as claimed in claim 1.

47. A toilet sanitiser comprising a composition as claimed in claim 1.

48. A diaper comprising a composition as claimed in claim 1.

49. A method of manufacturing a composition as claimed in claim 1 comprising contacting an adjuvant comprising a fungal cell or fragment thereof with a biocidally active compound, whereby the biocidally active compound is encapsulated by the adjuvant and retained passively therein.
